(19) United States
(12) Patent Application Publication
(10) Pub. No.: US 2006/0094698 A1
(43) Pub. Date: May 4, 2006

(54) Self-administered contraceptive injection of oily solution

(76) Inventors: Henrik De Nijs, Oss (NL); Hendrikus Adrianus Antonius Van Der Voort, Oss (NL); Dirk D. Leysen, Organon (NL)

Correspondence Address: INTERVET INC.;PATENT DEPARTMENT, PO BOX 318, MILLSBORO, DE, 19966-0318, US

(21) Appl. No.: 10/515,714

(22) PCT Filed: May 23, 2003
(86) PCT No.: PCT/EP03/50192

§ 371 Date: Aug. 30, 2005

(30) Foreign Application Priority Data
May 30, 2002 (EP) 02077126.7

(51) Int. Cl. A61K 31/56; A61M 31/00

(52) U.S. Cl. 514/170; 604/500

(57) ABSTRACT

The subject invention provides a pharmaceutical formulation in the form of an oily solution for injection to a subject comprising a contraceptively and/or therapeutically effective amount of a long-acting progestogen and a contraceptively and/or therapeutically effective amount of a long-acting androgen dissolved in a pharmaceutically acceptable oily medium wherein the injection is administered by the subject itself with a needle-less device, a mini-needle device or a pre-filled subcutaneous syringe.

### FIELD OF THE INVENTION

The subject invention concerns the field of (male and female) contraception and (male and female) hormone replacement therapy (HRT).

### BACKGROUND

Contraceptive methods for men and women are important for worldwide reproductive health.

However, no effective and efficient methods of male contraception are as of yet available.

Male contraception seeks to suppress spermatogenesis through the suppression of the gonadotropins luteinizing hormone (LH) and follicle-stimulating hormone (FSH). This results in a depletion of intratesticular testosterone and cessation of spermatogenesis.

Administration of progestagen results in a dose dependent suppression of pituitary gonadotrophins and consequently, a decrease in testosterone levels and a reversible inhibition of spermatogenesis. An exogenous androgen is required to compensate for the reduced testosterone levels. In the same way, male HRT can be accomplished, resulting in replacement of testosterone by an exogenous androgen which is safer on the prostate than endogenous testosterone.

The use of progestogens together with androgens for use as male contraceptives is known (Guerin and Rollet (1988), International Journal of Andrology 11, 187-199).

However, the use of specific esters of etonogestrel for male contraception and HRT has not been suggested.

In addition, the use of progestogens together with estrogens for use in female contraception is known (M. Tausk, J. H. H. Thijssen, Tj. B. van Wimersma Greidanus, “Pharmakologie der Hormone”, Georg Thieme Verlag, Stuttgart, 1986).

Progestagens are widely used for female contraception and in female HRT. In contraception, the combination progestagen-estrogen oral contraceptives are the most widely used. Administration of such a combination results in a number of effects: it blocks ovulation, it interferes with phasic development of the endometrium which decreases the chance for successful implantation, and it causes the cervical mucus to become so viscous that it hinders sperm penetration. Most progestagen-only-pills (POP's) aim at the last mentioned effect only.

Female HRT is aimed at suppletion of endogenous estrogen for the treatment of peri- and postmenopausal complaints (hot flushes, vaginal dryness), and for prevention of symptoms of long-term estrogen deficiency. The latter include osteoporosis, coronary artery disease, urogenital incontinence, and possibly also Alzheimer's disease and colorectal cancer. A drawback of long-term unopposed estrogen administration is the associated increase in endometrium proliferation, which in turn may increase the risk of endometrial cancer. For that reason, progestagens are co-administered in long-term regimes, because of their ability to reduce the proliferative activity of endometrial epithelium and to induce secretory conversion.

However, the use of specific esters of etonogestrel for female contraception, female HRT and treatment/prevention of gynaecological disorders has not been suggested.

There are also no disclosures of a male or female contraceptive/HRT solution for self-injection which would result in high compliance levels. Compliance is probably the most important factor in contraceptive use; without good compliance even the best contraceptives are without effect.

There is therefore a need for a male and female contraceptive which will have a high compliance rate in male and female subjects undergoing contraception.

High compliance depends on infrequent, painless administration without side effects and without local site reactions.

### SUMMARY OF THE INVENTION

The subject invention provides a pharmaceutical formulation in the form of an oily solution for injection to a subject comprising a contraceptively and/or therapeutically effective amount of a long-acting progestogen and a contraceptively and/or therapeutically effective amount of a long-acting androgen dissolved in a pharmaceutically acceptable oily medium wherein the injection is administered by the subject itself with a needle-less device, a mini-needle device or a pre-filled subcutaneous syringe and wherein the injectable volume of the solution is less than 1 milliliter.

The subject invention further contemplates a use of a long-acting progestogen and a long-acting androgen dissolved in a pharmaceutically acceptable oily medium for the manufacture of an injectable pharmaceutical formulation for male contraception wherein the injection is administered with a needle-less device, a mini-needle device or a pre-filled subcutaneous syringe and wherein the injectable volume of the solution is less than 1 milliliter.

The subject invention also provides a male contraceptive kit for injection comprising a long-acting progestogen and a long-acting androgen dissolved in an oily medium wherein the injection is administered by the subject itself with a needle-less device, a mini-needle device or a pre-filled subcutaneous syringe and wherein the injectable volume of the solution is less than 1 milliliter.

The subject invention further contemplates a method of male contraception comprising injecting a solution comprising a contraceptively and/or therapeutically-effective amount of a long-acting progestogen and a contraceptively and/or therapeutically effective amount of a long-acting androgen dissolved in an oily medium to a subject wherein the injection is administered by the subject itself with a needle-less device, a mini-needle device or a pre-filled subcutaneous syringe and wherein the injectable volume of the solution is less than 1 milliliter.

The subject invention also contemplates a pharmaceutical formulation in the form of an oily solution for injection to a subject comprising a contraceptively and/or therapeutically effective amount of a long-acting progestogen and a contraceptively and/or therapeutically effective amount of an estrogen dissolved in a pharmaceutically acceptable oily medium wherein the injection is administered by the subject itself with a needle-less device, a mini-needle device or a pre-filled subcutaneous syringe.

### FIGURES

FIG. 1

Chemical structures of etonogestrel heptanoate (etonogestrel enanthate), etonogestrel nonanoate, etonogestrel decanoate, etonogestrel undecanoate, etonogestrel dodecanoate, etonogestrel tridecanoate, and etonogestrel pentadecanoate.

FIG. 2a

Effect of one intramuscular (IM) injection of etonogestrel, etonogestrel heptanoate (etonogestrel enanthate), etonogestrel nonanoate and etonogestrel undecanoate on plasma levels of etonogestrel in male intact rabbits. Means and SEM of N=3.

FIG. 2b

Effect of one intramuscular (IM) injection of etonogestrel heptanoate (etonogestrel enanthate), etonogestrel nonanoate, etonogestrel decanoate, etonogestrel undecanoate, etonogestrel dodecanoate, etonogestrel tridecanoate on plasma levels of etonogestrel in male intact rabbits. Means and SEM of N=3.

FIG. 3

Chemical structure of MENT-undecanoate, MENT-buciclate, testosterone heptanoate (testosterone enanthate) and testosterone undecanoate.

FIG. 4

Time dependent effects of one s.c injection of 20 mg/kg of MENT-undecanoate (MENT-U), MENT-buciclate (MENT-B), testosterone heptanoate (testosterone enanthate, TE) and testosterone undecanoate (TU) in castrated male rabbits on serum MENT or testosterone (T). Results are means of N=3.

FIG. 5

Pharmacokinetics of testosterone enanthate, testosterone undecanoate and testosterone buciclate after one IM injected in male hypogonadal men with indicated doses on the plasma levels of serum testosterone. Normal range of serum testosterone is indicated with a dashed line. Derived from E. Nieschlag and H. M. Behre. Testosterone Therapy. In: Andrology, Male reproductive health and dysfunction., edited by E. Nieschlag and H. M. Behre, Berlin, Heidelberg and New York:Springer-Verlag, 1997, p. 297-309.

FIG. 6: completeness of injection

FIG. 7: pain scale

FIG. 8: immediate pain scores

FIG. 9: injection sensation scale

FIG. 10: injection sensation

FIG. 11: local site reactions after 2 hours

FIG. 12: local site reactions after 24 hours

FIG. 13: local site reactions after 5-7 days

FIG. 14: subject preference

### DETAILED DESCRIPTION OF THE INVENTION

The subject invention provides a pharmaceutical formulation in the form of an oily solution for injection to a subject comprising a contraceptively and/or therapeutically effective amount of a long-acting progestogen and a contraceptively and/or therapeutically effective amount of a long-acting androgen dissolved in a pharmaceutically acceptable oily medium wherein the injection is administered by the subject itself with a needle-less device, a mini-needle device or a pre-filled subcutaneous syringe and wherein the injectable volume of the solution is less than 1 milliliter.

The subject invention further contemplates a use of a long-acting progestogen and a long-acting androgen dissolved in a pharmaceutically acceptable oily medium for the manufacture of an injectable pharmaceutical formulation for male contraception wherein the injection is administered with a needle-less device, a mini-needle device or a pre-filled subcutaneous syringe and wherein the injectable volume of the solution is less than 1 milliliter.

The subject invention also provides a male contraceptive kit for injection comprising a long-acting progestogen and a long-acting androgen dissolved in an oily medium wherein the injection is administered by the subject itself with a needle-less device, a mini-needle device or a pre-filled subcutaneous syringe and wherein the injectable volume of the solution is less than 1 milliliter.

The subject invention further contemplates a method of male contraception comprising injecting a solution comprising a contraceptively and/or therapeutically-effective amount of a long-acting progestogen and a contraceptively and/or therapeutically effective amount of a long-acting androgen dissolved in an oily medium to a subject wherein the injection is administered by the subject itself with a needle-less device, a mini-needle device or a pre-filled subcutaneous syringe and wherein the injectable volume of the solution is less than 1 milliliter.

Similarly, a pharmaceutical formulation in the form of an oily solution for injection to a subject can be prepared comprising a contraceptively and/or therapeutically effective amount of a long-acting progestogen and a contraceptively and/or therapeutically effective amount of a long-acting estrogen dissolved in a pharmaceutically acceptable oily medium wherein the injection is administered by the subject itself with a needle-less device, a mini-needle device or a pre-filled subcutaneous syringe and wherein the injectable volume of the solution is less than 1 milliliter.

In a preferred embodiment, the long acting progestogen is an ester with a fatty chain length of C7 to C15, preferably an ester of a progestogen selected from the group consisting of ethisterone, norethisterone (norethindrone), dimethisterone, norethynodrel, norgestrienone, lynestrenol, ethynodiol, (levo)norgestrel, desogestrel, gestodene, allylestrenol, etonogestrel and dienogest. In a specific embodiment, the progestogen is an ester of etonogestrel with a fatty chain length of C10 to C12.

In a preferred embodiment, the long-acting androgen is an ester with a fatty chain length of C6 to C12, preferably an ester of testosterone or an ester of 7-alpha-methyl-19-nortestosterone (MENT). In a specific embodiment, the ester of 7-alpha-methyl-19-nortestosterone (MENT) is MENT undecanoate.

In a preferred embodiment, it is contemplated that the long-acting progestogen is an ester of etonogestrel and the long-acting androgen is an ester of 7-alpha-methyl-19-nortestosterone (MENT). In a most preferred embodiment, the ester of 7-alpha-methyl-19-nortestosterone (MENT) is MENT undecanoate and the ester of etonogestrel is etonogestrel undecanoate and/or etonogestrel decanoate and/or etonogestrel dodecanoate.

It is contemplated that the injection takes place once per month or once per two months.

The progestogen and testosterone esters can be prepared by dissolving it in a suitable amount of an oily medium, such as arachis oil, oleic acid, castor oil, ethyl undecanoate, almond oil, sesame oil, coconut oil, olive oil, soyabean oil, (purified) tri-glycerised, propylene glycol esters, ethyl oleate and the like, including mixtures of oils. The amount of esters that can be dissolved differs per chosen medium, but will generally be within the range of from 100-400 mg.

In a preferred embodiment it is further contemplated that the oily medium is arachis oil or ethyl undecanoate.

In a further embodiment, the contraceptively and/or therapeutically effective amount of MENT undecanoate is 50-400 mg and the contraceptively and/or therapeutically effective amount of etonogestrel ester is 25-200 mg. In a more specific embodiment, the contraceptively and/or therapeutically effective amount of MENT undecanoate is 50-200 mg and the contraceptively and/or therapeutically effective amount of etonogestrel ester is 50-100 mg. In a very specific embodiment, the contraceptively and/or therapeutically effective amount of MENT undecanoate is 100 mg and the contraceptively and/or therapeutically effective amount of etonogestrel ester is 50 mg.

Additives common to injection fluids can be added to the solution if desired. Suitable additives are known to the person skilled in the art. Possible additives include liquids that serve to lower the viscosity of the formulation, e.g. benzyl alcohol, benzyl benzoate, benzyl propionate, ethyl oleate or ethyl undecanoate. The present invention is further described in the following examples which are not in any way intended to limit the scope of the invention as claimed.

### EXAMPLES

### Example 1

### Kinetics of Etonogestrel C7, C9, C10, C11, C12 and C13 Esters in Rabbits

The following etonogestrel esters were prepared and tested in rabbits:
Etonogestrel heptanoate
Etonogestrel nonanoate
Etonogestrel decanoate
Etonogestrel undecanoate
Etonogestrel dodecanoate
Etonogestrel tridecanoate

Etonogestrel pentadecanoate was also prepared.

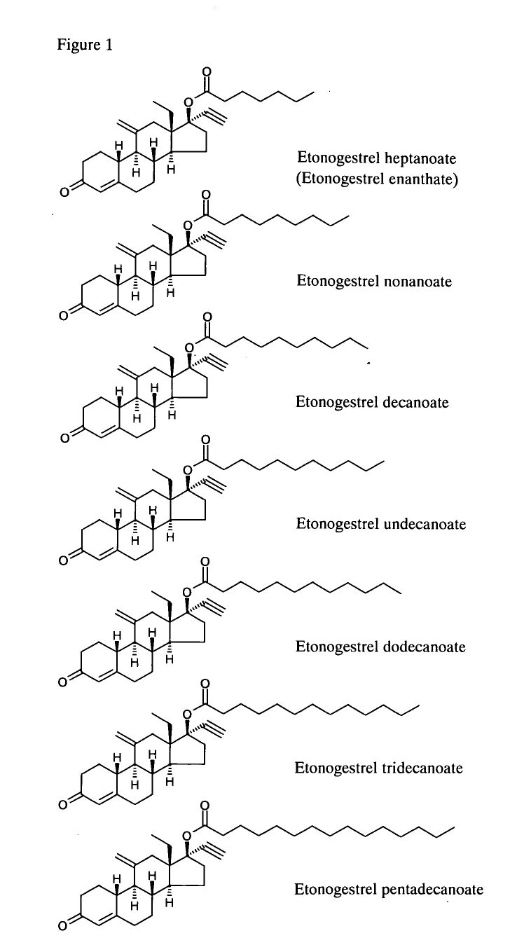

FIG. 1 shows the chemical structure of these compounds.

As a reference, etonogestrel was also included.

### Preparation of Etonogestrel Esters

General methodology for the preparation of esters from alcohols can be found in e.g. Greene, T. W. et al, “Protective groups in organic synthesis”, John Wiley & Sons, NY, 1999 (third edition). Preparation of esters from tertiary alcohols (like etonogestrel) can be accomplished by several techniques, for instance:

1) tertiary alcohol, carboxylic acid, trifluoroacetic acid-anhydride, DE 1013284 (1956); 2) tertiary alcohol, acid chloride, pyridine, Watson, T. G. et al, Steroids 41, 255 (1983); 3) tertiary alcohol, acid chloride, TIOEt, Shafiee, A. et al, Steroids 41, 349 (1983), 4) tertiary alcohol, carboxylic acid-anhydride, TsOH, benzene, Johnson, A. L., Steroids, 20, 263 (1972); and 5) tertiary alcohol, carboxylic acid-anhydride, DMAP, CH₂Cl₂, Shafiee, A. et al, Steroids 41, 349 (1983).

### Preparation of (17α)-13-Ethyl-11-methylene-17-[[(1-oxononyl)oxy]-18,19-dinorpregn-4-en-20-yn-3-one (etonogestrel nonanoate)

a) A solution of nonanoic acid (1.95 g) in dry toluene (8 ml) was cooled to 0° C. and treated with trifluoroacetic acid anhydride (2.6 g). After 30 min. stirring, (17α)-13-ethyl-17-hydroxy-11-methylene-18,19-dinorpregn-4-en-20-yn-3-one (etonogestrel, 2.0 g) in dry toluene (15 ml) was added and the reaction mixture was stirred for 17 h at room temperature. The reaction mixture was washed with water, a saturated aqueous solution of sodium hydrogen carbonate, water, and brine. The organic phase was dried over sodium sulfate and concentrated under reduced pressure. The residue was purified by column chromatography (toluene/ethyl acetate 95:5). The product (2.08 g) was dissolved in ethyl acetate (40 ml), cooled to 0° C., and stirred with aqueous sodium hydroxide (1 M, 13 ml) for 2 h. The mixture was extracted with ethyl acetate; the combined organic phases were washed with ice-cold aqueous sodium hydroxide (1 M), water and brine, dried and concentrated under reduce pressure. Column chromatography afforded (17α)-13-ethyl-11-methylene-17-[[(1-oxononyl)oxy]-18,19-dinorpregn-4-en-20-yn-3-one (1.25 g). ¹H-NMR (CDCl₃): δ 5.89 (m, 1H), 5.08 (bs, 1H), 4.85 (bs, 1H), 2.82 (ddd, 1H, J=14.8, 9.5 and 6.3 Hz), 2.73 (d, 1H, J=12.8 Hz), 2.69-2.19 (m), 2.63 (s, 1H), 2.11 (m, 1H), 1.90-1.21 (m), 1.15 (m, 1H), 1.05 (t, 3H, J=7.5 Hz), 0.88 (t, 3H, J=7.1 Hz). Measured mass [M+H]⁺465.3358. Calculated mass [M+H]⁺465.3363.

In a manner analogous to the procedure described above, etonogestrel heptanoate, etonogestrel decanoate, etonogestrel undecanoate, etonogestrel dodecanoate, etonogestrel tridecanoate, and etonogestrel pentadecanoate were prepared:
b) (17α)-13-Ethyl-11-methylene-17-[[(1-oxoheptyl)oxy]-18,19-dinorpregn-4-en-20-yn-3-one (etonogestrel heptanoate). ¹H-NMR (CDCl₃): δ 5.89 (m, 1H), 5.08 (bs, 1H), 4.85 (bs, 1H), 2.82 (ddd, 1H, J=14.8, 9.5 and 6.3 Hz), 2.73 (d, 1H, J=12.6 Hz), 2.68-2.19 (m), 2.63 (s, 1H), 2.11 (m, 1H), 1.90-1.24 (m), 1.15 (m, 1H), 1.05 (t, 3H, J=7.5 Hz), 0.89 (t, 3H, J=7.1 Hz). Measured mass [M+H]⁺437.3027. Calculated mass [M+H]⁺437.3050.
c) (17α)-13-Ethyl-11-methylene-17-[[(1-oxodecyl)oxy]-18,19-dinorpregn-4-en-20-yn-3-one (etonogestrel decanoate). ¹H-NMR (CDCl₃): δ 5.89 (bs, 1H), 5.08 (bs, 1H), 4.84 (bs, 1H), 2.82 (m, 1H), 2.73 (d, 1H, J=12.6 Hz), 2.67-2.18 (m), 2.63 (s, 1H), 2.11 (m, 1H), 1.90-1.21 (m), 1.15 (m, 1H), 1.06 (t, 3H, J=7.5 Hz), 0.88 (t, 3H, J=7.1 Hz). Measured mass [M+H]⁺479.3508. Calculated mass [M+H]⁺479.3519.
d) (17α)-13-Ethyl-11-methylene-17-[[(1-oxoundecyl)oxy]-18,19-dinorpregn-4-en-20-yn-3-one (etonogestrel undecanoate). ¹H-NMR (CDCl₃): δ 5.89 (m, 1H), 5.08 (bs, 1H), 4.85 (bs, 1H), 2.82 (ddd, 1H, J=14.8, 9.5 and 6.3 Hz), 2.73 (d, 1H, J=12.6 Hz), 2.68-2.18 (m), 2.63 (s, 1H), 2.11 (m, 1H), 1.90-1.21 (m), 1.06 (t, 3H, J=7.5 Hz), 0.88 (t, 3H, J=7.1 Hz). Measured mass [M+H]⁺493.3664. Calculated mass [M+H]⁺493.3676.
e) (17α)-13-Ethyl-11-methylene-17-[[(1-oxododecyl)oxy]-18,19-dinorpregn-4-en-20-yn-3-one (etonogestrel dodecanoate). ¹H-NMR (CDCl₃): δ 5.89 (bs, 1H), 5.08 (bs, 1H), 4.85 (bs, 1H), 2.82 (m, 1H), 2.73 (d, 1H, J=12.6 Hz), 2.65-2.18 (m), 2.64 (s, 1H), 2.11 (m, 1H), 1.90-1.20 (m), 1.15 (m, 1H), 1.06 (t, 3H, J=7.5 Hz), 0.88 (t, 3H, J=7.1 Hz). Measured mass [M+H]⁺507.3829. Calculated mass [M+H]⁺507.3832.
f) (17α)-13-Ethyl-11-methylene-17-[[(1-oxotridecyl)oxy]-18,19-dinorpregn-4-en-20-yn-3-one (etonogestrel tridecanoate). ¹H-NMR (CDCl₃): δ 5.89 (bs, 1H), 5.08 (bs, 1H), 4.85 (bs, 1H), 2.82 (m, 1H), 2.73 (d, 1H, J=12.6 Hz), 2.65-2.18 (m), 2.64 (s, 1H), 2.11 (m, 1H), 1.90-1.20 (m), 1.15 (m, 1H), 1.06 (t, 3H, J=7.5 Hz), 0.89 (t, 3H, J=7.1 Hz). Measured mass [M+H]⁺521.4007. Calculated mass [M+H]⁺521.3989.
g) (17α)-13-Ethyl-11-methylene-17-[[(1-oxopentadecyl)oxy]-18,19-dinorpregn-4-en-20-yn-3-one (etonogestrel pentadecanoate). ¹H-NMR (CDCl₃): δ 5.89 (bs, 1H), 5.08 (bs, 1H), 4.85 (bs, 1H), 2.82 (m, 1H), 2.73 (d, 1H, J=12.6 Hz), 2.65-2.19 (m), 2.63 (s, 1H), 2.11 (m, 1H), 1.90-1.20 (m), 1.15 (m, 1H), 1.06 (t, 3H, J=7.5 Hz), 0.89 (t, 3H, J=7.1 Hz). Measured mass [M+H]⁺549.4278. Calculated mass [M+H]⁺549.4302.
   Pharmacokinetics Studies in the Rabbit

For the determination of the pharmacokinetic profile of the different etonogestrel-esters after parenteral application, i.m. application in the castrated rabbit model was chosen instead of s.c. Briefly, rabbits were injected once (day 1) with indicated etonogestrel-esters at 20 mg/kg in arachis oil (with a concentration of 40 mg/ml). At day 1, 2, 3, 4, 5, 6, 7, 8, 10, 12, 14, 21, 28, 35, 49, 63, 77, 92, 106, 120 and 133 blood was collected from the ear arteria, in EDTA-containing tubes. EDTA plasma was prepared (1500 g, 15 min) and stored at −20° C. With LC-MSMS, the amount of parent compound (etonogestrel) was determined in these samples. The lower limit of this new assay is 0.5 nmol/l, from 0-250 nmol/l a linear curve was obtained with a correlation coefficient of 0.9998.

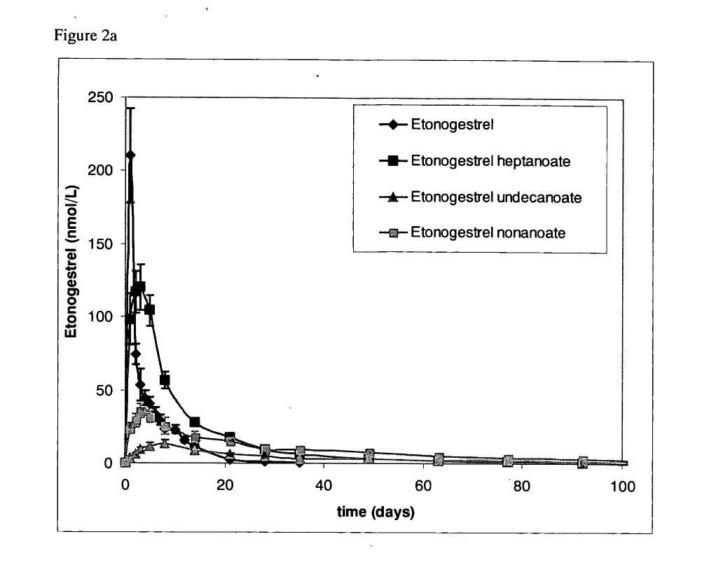

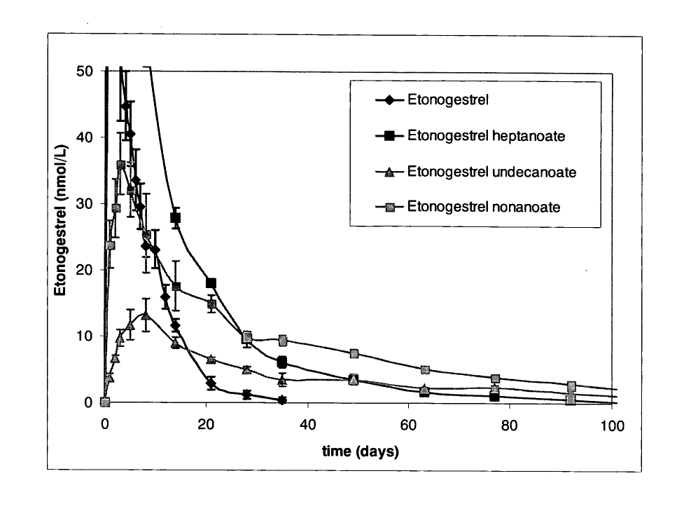

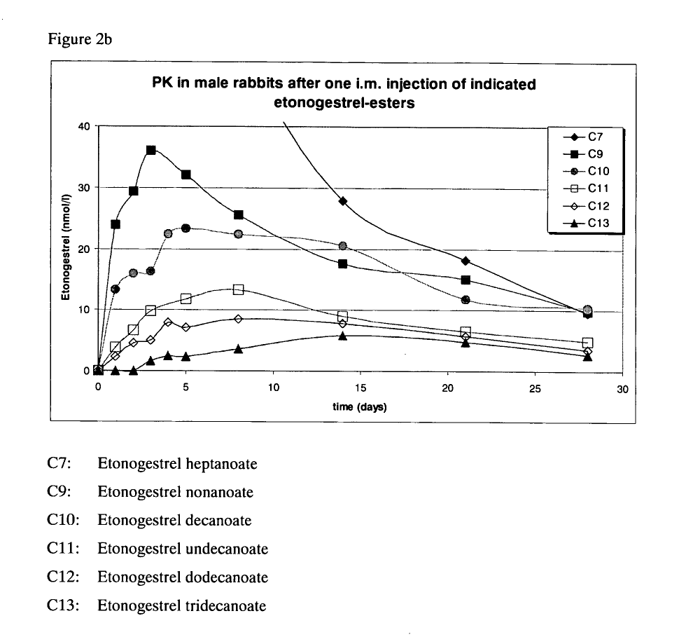

As shown in FIG. 2a, etonogestrel itself resulted in very high peak levels (200 nmol/l), which declined in 28 days to levels of etonogestrel below 1 nmol/l. Etonogestrel-heptanoate also gave rise to high initial peak levels of etonogestrel (120 nmol/l). Etonogestrel-nonanoate gave lower peak levels and extended duration with serum levels of etonogestrel above 1 nmol/l. As compared to the other two esters in FIG. 2a, etonogestrel undecanoate gave the most optimal balance between initial peak levels (maximum of 13 nmol/l after eight days) and duration of action (more than 92 days above 1 nmol/l).

As shown in FIG. 2b, etonogestrel decanoate gave an initial peak level of 24 nmol/l after 5 days whereas etonogestrel dodecanoate gave an initial peak level of 9 nmol/l after 8 days. With etonogestrel tridecanoate, no initial levels of etonogestrel were observed.

From FIGS. 2a and 2b, it can be seen that preferred etonogestrel esters are etonogestrel decanoate, etonogestrel undecanoate, and etonogestrel dodecanoate.

### Example 2

### Kinetics of Two MENT Esters in Rabbits

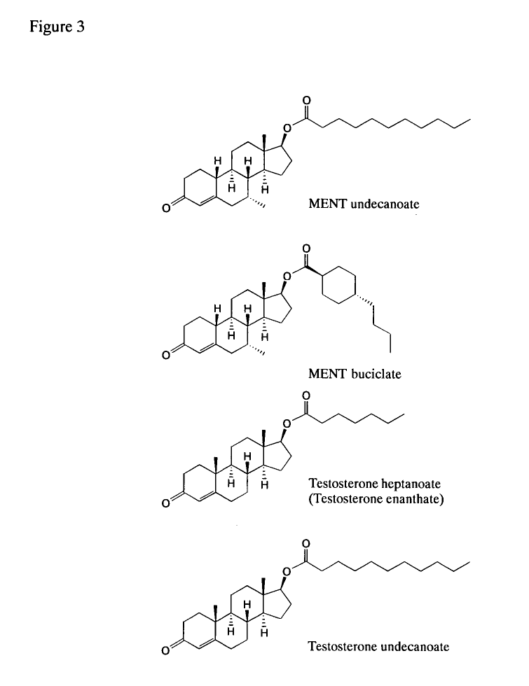

The pharmacokinetic profile of MENT-undecanoate and MENT-buciclate was compared to testosterone enanthate and testosterone undecanoate. FIG. 3 shows the chemical structures of these androgen esters.

Ment-undecanoate was prepared essentially as described in WO 99/67271. MENT-buciclate was prepared as described in WO 99/67270. Testosterone enanthate and undecanoate were commercially obtained from Diosynth, Oss, the Netherlands.

Pharmacokinetic Studies in the Rabbit

For the determination of the pharmacokinetic profile of the different androgen-esters after s.c. application, the castrated rabbit model was selected as the model which is most similar to humans. Briefly, rabbits were injected once (day 1) with indicated androgen-esters at 20 mg/kg in arachis oil (with a concentration of 100 mg/ml). At day 2, 3, 4, 5, 8, 15, 22, 36, 44 and 58 blood was collected from the ear arteria, in EDTA-containing tubes. EDTA plasma was prepared (1500 g, 15 min) and stored at −20° C. With LC-MSMS, the amount of parent compound (testosterone or MENT) was determined in these samples. The lower limit of this new assay is 2 nmol/l, from 0-500 nmol/I a linear curve was obtained with a correlation coefficient of 0.9998.

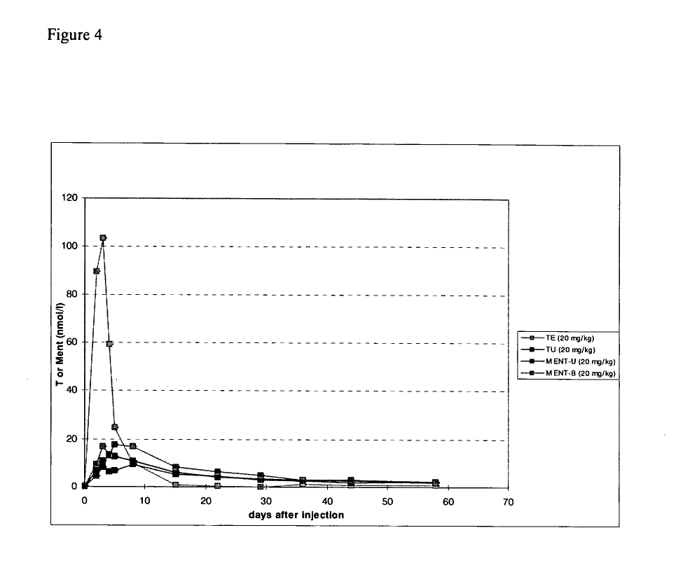

As shown in FIG. 4, both with MENT-undecanoate and MENT-buciclate a pharmacokinetic profile of released MENT was found which is similar to that of the reference compound testosterone undecanoate with respect to released testosterone. Testosterone enanthate resulted in a high peak of testosterone 2 days after injection.

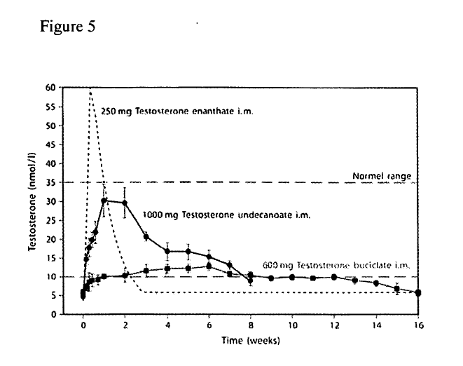

Thus, in the rabbit, with both MENT-esters no initial rise of MENT was observed on one hand and a prolonged release of MENT was observed on the other hand, suggestive for more optimal pharmacokinetic behaviour than the current standard testosterone enanthate. In humans, optimal pharmacokinetics were obtained with testosterone undecanoate: low initial release and steady-state levels of long duration (FIG. 5). Since in rabbits the pharmacokinetic profile of the two MENT-esters was very similar to that of testosterone-undecanoate (FIG. 4), optimal pharmacokinetics with both MENT esters in humans is expected.

### Example 3

### Solubility and Viscosity of MENT-Undecanoate and Etonogestrel Undecanoate in Various Solvents

To determine the solubility and viscosity of MENT undecanoate and etonogestrel undecanoate, four different solvents were used:
ethyl undecanoate
ethyl undecanoate+50% benzyl benzoate
arachis oil
arachis oil+50% benzyl benzoate

Using these solvents, the following solutions were prepared:
100 mg/ml etonogestrel undecanoate in the different solvents
50 mg/ml etonogestrel undecanoate in the different solvents
200 mg/ml MENT undecanoate in the different solvents
100 mg/ml MENT undecanoate in the different solvents
50 mg/ml etonogestrel undecanoate+100 mg/ml MENT undecanoate in the different solvents

The two combined solvents were prepared by addition of 50 gram of ethyl undecanoate or arachis oil to 50 gram of benzyl benzoate. The ethyl undecanoate+50% benzyl benzoate solution was filtered over a 0.22 μm Durapore filter to obtain a clear colourless solution. The arachis oil+50% benzyl benzoate solution was not filtered.

The solubility of the compounds in the solvents was determined visually. The viscosity was determined using a Brookfield model DV-III. The density of the solutions was determined using a Mettler Toledo DA-100M density meter.

TABLE 1
Appearance, viscosity and density of the solvents

| Solvent | Appearance | Viscosity | Density |
|---|---|---|---|
| Ethyl undecanoate | Clear colourless solution | 2.6 | 0.861 |
| Ethyl undecanoate + | Clear colourless solution | 3.9 | 0.975 |
| 50% benzyl benzoate | | | |
| Arachis oil | Clear yellowish solution | 64.1 | 0.913 |
| Arachis oil + 50% | Clear yellowish solution | 22.9 | 1.007 |
| benzyl benzoate | | | |
| Benzyl benzoate | Clear yellowish solution | 8.5 | 1.117 |

Ethyl undecanoate, ethyl undecanoate+50% benzyl benzoate and arachis oil+50% benzyl benzoate solutions did not need to be heated. To dissolve 200 mg/ml MENT undecanoate in arachis oil, heating to approximately 50° C. was necessary.

The concentrations tested were 100 mg/ml etonogestrel undecanoate, 200 mg/ml MENT undecanoate and 50 mg/ml etonogestrel undecanoate+100 mg/ml MENT undecanoate in the different solvents. The results are summarized in table 2.

TABLE 2
Appearance, viscosity and density of the final solutions

| | Etonogestrel | MENT | | | |
|---|---|---|---|---|---|
| | undecanoate | undecanoate | | Viscosity | Density |
| Solvent | (mg/ml) | (mg/ml) | Appearance | (cps) | (g/ml) |
| Ethyl undecanoate | 50 | — | Clear colourless solution | 3.2 | 0.870 |
| | — | 100 | Clear colourless solution | 4.0 | 0.879 |
| | 50 | 100 | Clear colourless solution | 4.4 | 0.886 |
| Ethyl undecanoate + | 50 | — | Clear colourless solution | 4.7 | 0.978 |
| 50% benzyl benzoate | — | 100 | Clear colourless solution | 6.1 | 0.979 |
| | 50 | 100 | Clear colourless solution | 7.0 | 0.979 |
| Arachis oil | 50 | — | Clear yellowish solution | 76.6 | 0.919 |
| | — | 100 | Clear yellowish solution | 97.2 | 0.924 |
| | 50 | 100 | Clear yellowish solution | 99.7 | 0.935 |
| Arachis oil + 50% | 50 | — | Clear yellowish solution | 28.1 | 1.006 |
| benzyl benzoate | — | 100 | Clear yellowish solution | 35.0 | 1.009 |
| | 50 | 100 | Clear yellowish solution | 39.1 | 1.008 |

The combination of etonogestrel-undecanoate and MENT-undecanoate was visually dissolved at a desired concentration of 50 mg/ml etonogestrel-undecanoate and 100 mg/ml MENT-undecanoate in all four tested solvents. Both etonogestrel-undecanoate and MENT-undecanoate could be dissolved at two times the desired concentration in all four solvents tested. No precipitation occurred at room temperature when 50 mg/ml etonogestrel-undecanoate and 100 mg/ml MENT-undecanoate were dissolved in all four solvents.

The viscosity of ethyl undecanoate and ethyl undecanoate+50% benzyl benzoate was significantly lower than the viscosity of arachis oil and arachis oil+50% benzyl benzoate. The viscosity of the desired formulation 50 mg/ml etonogestrel undecanoate+100 mg/ml MENT undecanoate in the four different solvents was the lowest (4 cps) for the ethyl undecanoate solution, followed by the ethyl undecanoate+50% benzyl benzoate (7 cps) and the arachis oil+50% benzyl benzoate solution (39 cps). The viscosity of the arachis oil solution was significantly higher that the viscosity of the other solutions (100 cps).

### Example 4

### Pharmacological Action of Etonogestrel Esters in the Male

The pharmacological action of etonogestrel esters in the male are evaluated for the suppressing activity of endogenous testosterone in the rabbit as described in Wu, F. C., Balasubramanian, R., Mulders, T. M. and Coelingh-Bennink H. J., Oral progestogen combined with testosterone as a potential male contraceptive: additive effects between desogestrel and testosterone enanthate in suppression of spermatogenesis, pituitary-testicular axis, and lipid metabolism, J. Clin. Endocrinol. Metab 84 (1):112-122, 1999. Briefly, the effect of one sc/im injection of the different etonogestrel esters on serum testosterone at day 7 of mature male rabbits will be monitored.

### Example 5

### The Pharmacological Action of Etonogestrel Esters in the Female

The pharmacological action of etonogestrel esters in the female are tested in the classical Clauberg test. Briefly, immature female rabbits, primed with oestradiol for 8 days, are treated once sc/im with the different etonogestrel esters (day 8 afternoon). Autopsy is performed in the afternoon of day 13 and the progestagenic activity is evaluated on sections of the uterine according to McPhail et al., The assay of progestin. J. of Physiology, 1934, 83:145-156.

### Example 6

### Needle-Less Administration of Arachis Oil in Human Volunteers

Arachis oil was administered by a needle-less device and by needle and syringe to compare six parameters:

(1) completeness of injection; (2) injection pain; (3) injection sensation; (4) local site reactions; (5) subject preference; and (6) systemic adverse effects.

Forty-eight (48) healthy men aged 18-70 were recruited for an open-label, randomised, needle controlled trial. The men were divided into four groups:

Group 1: intramuscular injection with arachis oil and 10% benzyl alcohol with a needle and a syringe IM (1.5 inch, 20 gauge needle)—hereinafter called device A

Group 2: subcutaneous injection with arachis oil and 10% benzyl alcohol with a needle and a syringe S.C. (1.0 inch, 20 gauge needle)—hereinafter called device B

Group 3: intramuscular injection with arachis oil and 10% benzyl alcohol with the needle-less device Medi-Jector Needle Free System (MJ7) IM (100 lb. spring, 0.014 orifice (differential pressure)—hereinafter called device C

Group 4: subcutaneous injection with arachis oil and 10% benzyl alcohol with the needle-less device Medi-Jector Needle Free System (MJ7) S.C. (85 lb. spring, 0.011 orifice)—hereafter called device D

The men visited the clinic three times. During the first visit, the men were trained how to self-inject in two injection sessions with two injections each separated by 2 hours. Each session was either with IM or S.C. needles or MediJector. The injections were randomised to right or left and upper or lower thighs. The local site reaction (pain, itching, redness, swelling, bruising and sensation) was evaluated immediately after each injection and for two hours thereafter and a patient preference questionnaire was filled-out.

During the second visit, 24 hours later, the local site reaction and any adverse experiences were evaluated. During the third visit, 5-7 days later, local site reactions and adverse experiences were again evaluated.

Completeness of Injection

To assess the completeness of injection, the following penetration rating scale was used:

(1)—all of the oil penetrated the skin; (2S)—slight wetness on the skin; (2)—most of the oil penetrated the skin; (3)—about half the oil penetrated the skin; (4)—very little of the oil penetrated the skin.

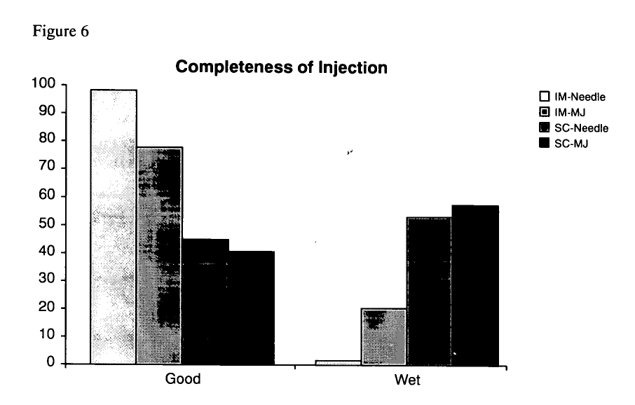

FIG. 6 shows the results. Most complete injection was achieved with the IM needle and thereafter with the IM MediJector (device A and C respectively).

Injection Pain

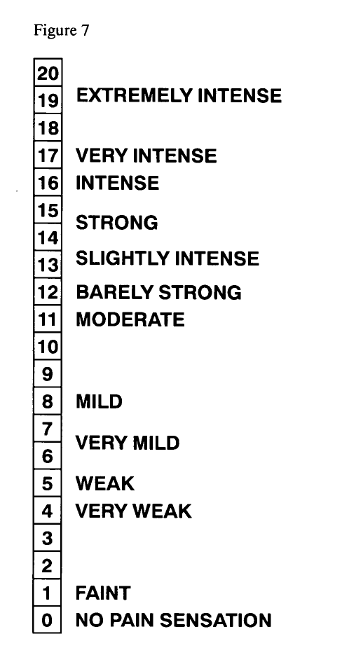

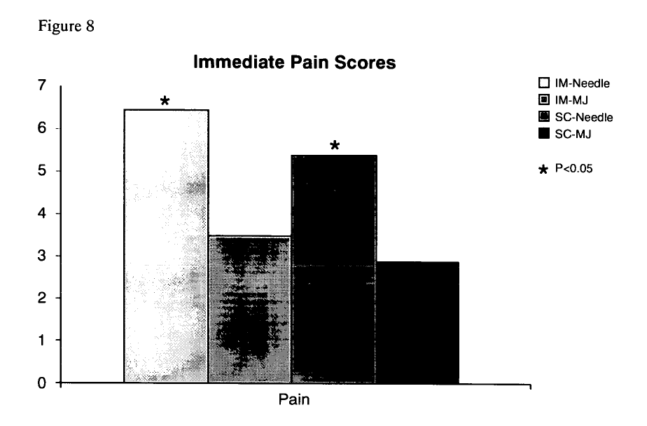

To assess pain, a pain scale was used (FIG. 7). FIG. 8 clearly shows that the least pain was experienced with the IM MediJector, and the most pain with the IM Needle.

Injection Sensation

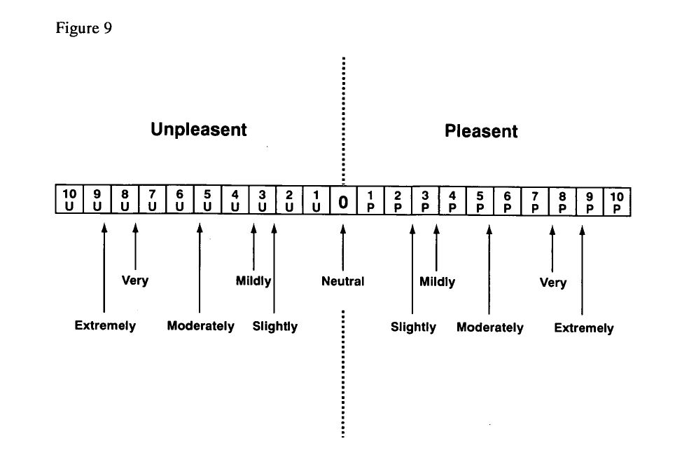

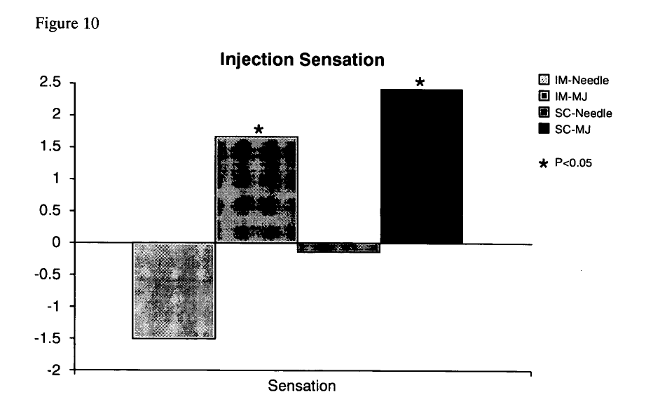

To assess injection sensation, a scale was used as presented in FIG. 9. FIG. 10 shows that both MediJector devices caused less injection sensation.

Local Site Reactions

To assess the local site reactions, the following 4-point evaluation scale was used: 0—no reaction; 1—mild reaction; 2—moderate reaction; 4—severe reaction

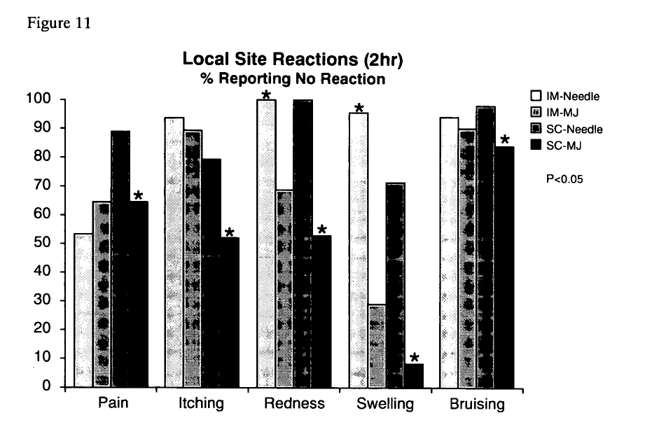

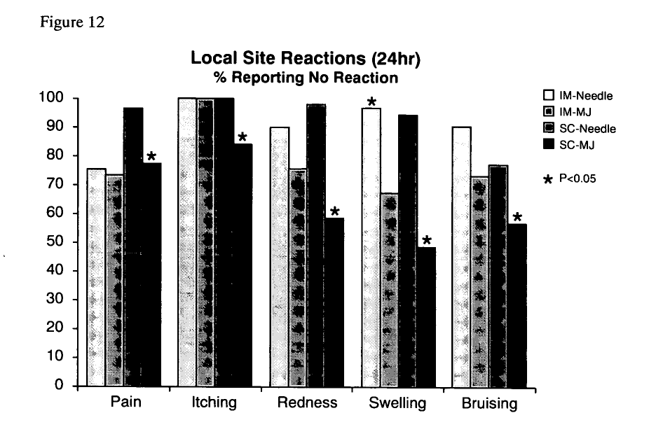

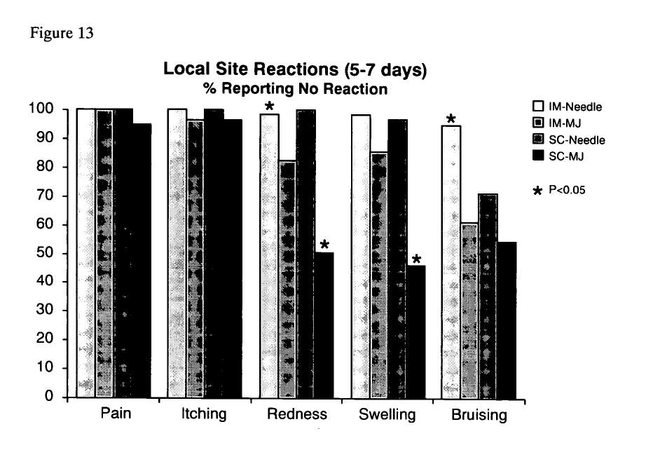

FIG. 11 shows the local site reactions after 2 hours, FIG. 12 after 24 hours and FIG. 13 after 5-7 days.

Subject Preference

The patient preference questionnaire included the following questions:

Question 1—Overall I found the injections for device A,B,C,D

—very unpleasant; —somewhat unpleasant; —slightly unpleasant; —hardly unpleasant; —not at all unpleasant.

Question 2—How willing would you be to have a doctor give you an injection with device A,B,C,D

—very willing; —somewhat willing; —neutral; —somewhat unwilling; —very unwilling.

Question 3—How willing would you be to give yourself an injection with device A,B,C,D

—very willing; —somewhat willing; —neutral; —somewhat unwilling; —very unwilling.

Question 4-which device would you be most willing to use to give yourself injections at home?

—IM Needle and Syringe (Device A); —S.C. Needle and Syringe (Device B); —IM-MediJector (Device C); S.C. MediJector (Device D).

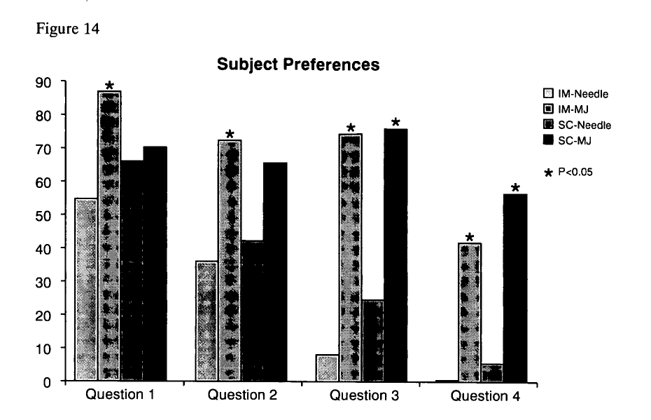

FIG. 14 shows the results of the questionnaire.

Systemic Adverse Events

In total 7 adverse events were reported: 2 blisters and 5 crusts at injection site. The events were all mild and involved all four devices. The events were probably related to the oil.

### CONCLUSIONS

The above trial shows that S.C. administration of oil has some percentage of wet injections.

IM and S.C. MediJectors were significantly less painful than needles. They were also considered more pleasant.

Even though MediJectors had a greater incidence of local site reactions (mild and clinically insignificant), subjects had a significant preference for the needle-free MediJector.

In order to achieve a higher completeness of injection with IM MediJector, the spring force can be increased. Another possibility is the use of a mini-needle device.

1-72. (canceled)

73. An oily solution for injection to a subject comprising a 50-400 mg/ml of a long-acting progestogen and 25-200 mg/ml of a long-acting androgen dissolved in a pharmaceutically acceptable oily medium.

74. A solution according to claim 1 wherein the long acting progestogen is an ester with a fatty chain length of C7 to C15.

75. A solution according to claim 2 wherein the long acting progestogen is an ester or a progestogen selected from the group consisting of ethisterone, norethisterone, dimethisterone, norethynodrel, norgestrienone, lynestrenol, ethynodiol, norgestrel, levo-norgestrel, desogestrel, gestodene, allylestrenol, etonogestrel and dienogest.

76. A solution according to claim 3 wherein the ester of progestogen is etonogestrel undecanoate.

77. A solution according to claim 1 wherein the long-acting androgen is an ester with a fatty chain length of C6 to C12.

78. A solution according to claim 5 wherein the long-acting androgen is an ester of testosterone or an ester of 7-alpha-methyl-19-nortestosterone.

79. A solution according to claim 6 wherein the ester of the androgen is 7-alpha-methyl-19-nortestostetone undecanoate.

80. A solution according to claim 1 wherein the oily medium is arachis oil or ethyl undecanoate.

81. A needle-less device, a mini-needle device or a pre-filled subcutaneous syringe, each for injection administration by the subject himself, comprising less than 1 millilitre of the solution according to claim 1.
